(19) United States
(12) Patent Application Publication
(10) Pub. No.: US 2024/0043377 A1
(43) Pub. Date: Feb. 8, 2024

(54) METHOD FOR DRYING METHYL MERCAPTAN BY MEANS OF AZEOTROPIC DISTILLATION

(71) Applicant: ARKEMA FRANCE, Colombes (FR)

(72) Inventors: Georges FREMY, Lacq (FR); Jean-Michel RAYMOND, Cauneille (FR)

(73) Assignee: ARKEMA FRANCE, Colombes (FR)

(21) Appl. No.: 18/256,693

(22) PCT Filed: Dec. 16, 2021
(86) PCT No.: PCT/FR2021/052364

§ 371 Date: Jun. 9, 2023

(30) Foreign Application Priority Data
Dec. 17, 2020 (FR) 2013436

(51) Int. Cl. C07C 319/28; C07C 319/08

(52) U.S. Cl. CPC C07C 319/28; C07C 319/08

(57) ABSTRACT

The present invention relates to a process for drying methyl mercaptan, notably by azeotropic distillation, comprising the following steps:
1) a stream (A) comprising methyl mercaptan and water is introduced into a distillation column (1);
2) said stream (A) is distilled in said column (1);
3) the distillate (B) is recovered in gaseous form, preferably at the top of the column;
4) the distillate (B) is condensed, preferably in a condenser (2), so as to obtain a condensate (C) in liquid form;
5) said condensate (C) is separated, preferably using a decanter (3), so as to obtain two separate liquid phases:
   an aqueous phase (D); and
   an organic phase (E) comprising methyl mercaptan;
6) all or part of the organic phase (E) is optionally introduced into the distillation column (1) as reflux; and
7) a stream (F) comprising the dried methyl mercaptan is recovered, preferably at the bottom of column (1).

The present invention also relates to processes for producing methyl mercaptan comprising said drying process.

The present invention relates to a process for drying methyl mercaptan, notably by azeotropic distillation. The present invention also relates to processes for producing methyl mercaptan comprising said drying process.

Mercaptans are of great interest industrially and are currently in widespread use in the chemical industries, notably as starting materials in the synthesis of more complex organic molecules. For example, methyl mercaptan (CH₃SH or MeSH) is used as a starting material in the synthesis of methionine, an essential amino acid for animal nutrition. Methyl mercaptan is also used in the synthesis of dialkyl disulfides, in particular in the synthesis of dimethyl disulfide (DMDS), a sulfiding additive for hydrotreating catalysts for petroleum fractions, among other applications.

The industrial synthesis of methyl mercaptan generally takes place according to two known routes. The first, called the methanol route, produces methyl mercaptan from methanol and hydrogen sulfide according to the following reaction (1):

CH₃OH+H₂S→CH₃SH+H₂O (1)

With this process, a side reaction gives rise to the formation of dimethyl sulfide, according to the following reaction (2):

CH₃OH+CH₃SH→CH₃SCH₃+H₂O (2)

The second route, called the carbon oxide route, makes it possible to obtain methyl mercaptan from a carbon oxide, hydrogen, hydrogen sulfide and/or sulfur, for example according to reactions (3) and (4) below:

CO+2H₂+H₂S→CH₃SH+H₂O (3)

CO+S+3H₂→CH₃SH+H₂O (4)

As indicated by the preceding reactions, the synthesis of methyl mercaptan is accompanied by a production of water irrespective of the route used. Thus, it is then necessary to separate the methyl mercaptan from the water. However, water is slightly soluble in methyl mercaptan. There is thus always some left in the product obtained, to be eliminated as much as possible.

Indeed, there are industrial applications for which a very low residual water content in methyl mercaptan is desirable. For example, in the synthesis of dimethyl disulfide by sulfur oxidation of methyl mercaptan, water can make the catalysts for this reaction less active.

In addition, when the residual water content in the methyl mercaptan is high (notably of the order of a few thousand ppm), and when the temperature is below approximately 16° C., part of the water can be desolubilized, decant and promote the formation of solid methyl mercaptan hydrates. These solid residues can lead to risks of clogging of equipment, leading to major safety problems for installations and transport.

To avoid these risks, the drying of methyl mercaptan is conventionally performed by adsorption of water on molecular sieves. However, this method has many drawbacks. For example, the regeneration of molecular sieves takes place at high temperatures and leads to the formation of undesirable byproducts such as dimethyl sulfide.

Furthermore, when methyl mercaptan is formed via the methanol route, traces of methanol in the methyl mercaptan to be dried can drastically reduce the water adsorption capacity of the molecular sieves. This entails increasing the frequency of regenerations, which increases production costs and the formation of undesirable byproducts.

There is thus a need for a methyl mercaptan with a low water content, preferably with the lowest possible water content.

There is also a need for a process for drying methyl mercaptan which is effective and which makes it possible to avoid all or some of the drawbacks of the known drying processes.

One object of the present invention is to provide a process for drying methyl mercaptan, which makes it possible to obtain a methyl mercaptan with a low water content, preferably with a water content of less than or equal to 1500 ppm.

Another object of the present invention is to provide a process which can totally or partly overcome the drawbacks of the drying processes used hitherto and notably using molecular sieves.

An object of the present invention is also to provide a process whose drying methods are controlled and/or which do not vary over time.

An object of the present invention is to provide a process for preparing methyl mercaptan which makes it possible to obtain a methyl mercaptan with a low water content, preferably with a water content of less than or equal to 1500 ppm.

An object of the present invention is to provide an integrated process for preparing methyl mercaptan, which is more environmentally friendly and more economical.

The present invention meets all or some of the above objectives.

Methyl mercaptan and water can form an azeotropic mixture, preferably a heteroazeotrope. The term “azeotropic mixture” notably means a liquid mixture which boils while keeping a fixed composition (the vapor phase has the same composition as the liquid phase). Preferably, methyl mercaptan and water form an azeotropic mixture at a pressure of between 0.05 and 75 bar absolute, preferably between 1 and bar absolute, more preferentially between 5 and 15 bar absolute.

Thus, the present inventors have discovered that distillation, preferably azeotropic, makes it possible to perform the drying of methyl mercaptan.

Surprisingly, the process according to the invention in fact makes it possible to effectively dry methyl mercaptan. In particular, the drying process according to the invention makes it possible to obtain a methyl mercaptan comprising between 0 and 1500 ppm of water.

Unlike molecular sieves, the drying process according to the invention also makes it possible to maintain drying methods which are not altered over time and which are easily controllable depending on the operating conditions, notably the temperature and pressure conditions. In particular, it is possible to control and/or choose the water content of the methyl mercaptan obtained by virtue of the drying process according to the invention.

Moreover, said drying process avoids the molecular sieve regeneration cycles and thus avoids the additional formation of dimethyl sulfide (DMS), an unwanted byproduct (sometimes incinerated as waste).

The drying process according to the invention is easy to implement and can be adapted to any installation for the production of methyl mercaptan, notably via the methanol or the carbon oxide route. It is thus possible to obtain an integrated process for the production of methyl mercaptan, which is more environmentally friendly and more economical.

According to the present invention, the unit ppm (part per million) refers to a mass fraction.

According to the present invention, the expression “between X and X” includes the limits described.

The term “drying” means the removal of water.

The term “dried methyl mercaptan” notably means methyl mercaptan resulting from the drying process according to the invention. Stream (F) defined below can comprise, or even consist of, said dried methyl mercaptan.

In particular, the term “dried methyl mercaptan” means a methyl mercaptan comprising between 0 and 1500 ppm, preferably between 0 and 1000 ppm, for example between 10 and 800 ppm, more preferentially between 40 and 800 ppm of water, relative to the total by weight of methyl mercaptan and water.

The term “dried methyl mercaptan” can also be understood to mean a composition comprising methyl mercaptan and between 0 and 1500 ppm, preferably between 0 and 1000 ppm, for example between 10 and 800 ppm, more preferentially between and 800 ppm of water, relative to the total by weight of methyl mercaptan and water.

Optionally, said dried methyl mercaptan may also include traces of methanol, H²S and sulfur byproducts. The term “traces” of a compound is understood to mean an amount of between 0 and 1000 ppm. In particular, the sulfur byproducts are dimethyl sulfide and dimethyl disulfide.

The dried methyl mercaptan may be in liquid or gaseous form, preferably in liquid form.

The term “methyl mercaptan purification step” notably means a step for producing a stream enriched in methyl mercaptan. The term “stream enriched in methyl mercaptan” notably means a stream which comprises a weight percentage of methyl mercaptan (relative to the total weight of said stream) greater than the weight percentage of methyl mercaptan relative to the total weight of said stream before said purification step.

Methyl Mercaptan Drying Process

The present invention relates to a process for drying methyl mercaptan, comprising the following steps:

1) a stream (A) comprising methyl mercaptan and water is introduced into a distillation column (1);
2) said stream (A) is distilled in said column (1);
3) the distillate (B) is recovered in gaseous form, preferably at the top of the column;
4) the distillate (B) is condensed, preferably in a condenser (2), so as to obtain a condensate (C) in liquid form;
5) said condensate (C) is separated, preferably using a decanter (3), so as to obtain two separate liquid phases:
   an aqueous phase (D); and
   an organic phase (E) comprising methyl mercaptan;
6) all or part of the organic phase (E) is optionally introduced into the distillation column (1) as reflux; and
7) a stream (F) comprising the dried methyl mercaptan is recovered, preferably at the bottom of the column (1).

Stream (F) corresponds notably to the dried methyl mercaptan as defined above. It is recovered from the distillation column, preferably at the bottom of the distillation column.

The distillation of step 2) may be performed at a pressure of between 0.05 and 75 bar absolute, preferably between 1 and 30 bar absolute, more preferentially between 5 and 15 bar absolute, for example at about 10, 11, 12, 13, 14 or 15 bar absolute.

The distillation of step 2) may be performed at a temperature of between 20° C. and 200° C., preferably between 60° C. and 100° C., more preferentially between 65° C. and for example between 70° C. and 90° C. Preferably, the distillation of step 2) may be performed at a temperature of between 40° C. and 200° C., preferably between 80° C. and 100° C. at the bottom of the column, and between 20° C. and 100° C., preferably between 60° C. and 80° C. at the top of the column.

Particularly preferably, the distillation of step 2) is performed at a pressure of between and 15 bar absolute and at a temperature of between 60° C. and 100° C. In particular, the distillation of step 2) is performed at a pressure of between 5 and 15 bar absolute and at a temperature of between 70° C. and 90° C. In particular, the distillation of step 2) is an azeotropic distillation.

The distillation of step 2) may be performed in any known type of distillation column. It may be a column with plates (for example plates with caps, plates with valves or perforated plates) or with packing (for example with bulk or structured packing). The distillation of step 2) may be performed in a plate column, preferably comprising between 5 and 50 plates, more preferentially between 10 and 40 plates, for example between 25 and 30 plates. The distillation of step 2) may also be performed in a partition column (“DWC” or Divided Wall Column). The partition may be fixed or mobile, for example with structured or bulk packing.

Stream (A) is preferably in liquid or gaseous form.

Preferably, stream (A) comprises, or even consists of, methyl mercaptan, water and optionally traces of methanol, H₂S and sulfur byproducts.

Stream (A) may comprise at least 90%, preferably at least 95%, more preferentially at least 98%, for example at least 99% by weight of methyl mercaptan, relative to the total by weight of methyl mercaptan and water.

Stream (A) may comprise at least 0.15% by weight of water, preferably at least strictly greater than 0.15% by weight of water, relative to the total weight of water and methyl mercaptan. Stream (A) may comprise a maximum of 30%, preferably a maximum of 10% by weight of water, relative to the total by weight of methyl mercaptan and water. Stream (A) may comprise between 0.15%, preferably strictly greater than 0.15%, and 30% by weight of water relative to the total by weight of methyl mercaptan and water. Stream (A) may comprise between 0.15%, preferably strictly greater than 0.15%, and 10% by weight of water relative to the total by weight of methyl mercaptan and water. Preferably, stream (A) comprises between 0.15%, preferably strictly greater than 0.15%, and 5% by weight of water relative to the total by weight of methyl mercaptan and water.

For example, stream (A) comprises between 0.15%, preferably strictly greater than and 2%, for example between 0.15% and 1.5% or between 0.15% and 1% in weight of water, relative to the total by weight of methyl mercaptan and water; the remainder possibly being methyl mercaptan.

Following step 2) of distillation of stream (A), a gaseous distillate (B) is obtained. This distillate (B) corresponds notably to an azeotropic mixture, preferably a heteroazeotropic mixture, in particular under the pressure and/or temperature conditions of distillation step 2).

Thus, the distillation of step 2) makes it possible notably to form an azeotropic mixture (i.e. an azeotropic distillation). Once recovered and condensed in liquid form (condensate (C)), it is found in two-phase form, the two phases of which can be readily separated, notably by decantation.

Step 4) of condensation of the distillate (B) may be performed via any conventional technique. The condensation may be performed in a condenser separate from the distillation column or which can be integrated into said column. A condensate (C) in liquid form is then obtained, preferably comprising two phases, one of which is aqueous and the other organic (and comprising the methyl mercaptan). During the condensation step 4), the temperature may be between 20° C. and 50° C. and/or the pressure may be between 5 and 15 bar absolute.

The distillate (B) and the condensate (C) preferably have the same composition.

During step 5) of separation, any known method may be used. Most preferably, decantation is used. During the separation step, the temperature may be between 20° C. and 50° C. and/or the pressure may be between 5 and 15 bar absolute. On conclusion of step 5), two separate liquid phases are obtained:
an aqueous phase (D); and
an organic phase (E) comprising methyl mercaptan.

According to one embodiment, the aqueous phase (D) comprises:
water,
H₂S, preferably in trace amounts,
possibly methyl mercaptan, preferably in trace amounts; and
possibly sulfur byproducts, preferably in trace amounts.

The H₂S, and possibly methyl mercaptan and sulfur byproducts, can be separated from said aqueous phase. The separation may be performed via any known means and preferably by stripping, which may be thermal stripping or by stripping with inert gas (for example by stripping with nitrogen, methane or CO₂). This gaseous phase then forms vents called vents E3 below.

According to one embodiment, the vents E3 are incinerated and/or the aqueous phase (D) can be eliminated in the waste water network.

According to another embodiment, the vents E3 can be sent to a methanol absorption column, in order to recover the sulfur compounds such as H₂S and/or methyl mercaptan which they include, by gas(vent)-liquid(methanol) extraction.

According to one embodiment, the organic phase (E) is recovered on conclusion of step 5) when the reflux step 6) is not performed.

According to another embodiment, the organic phase (E) is used totally or partly as reflux of the distillation column (1).

In step 6), the reflux ratio may be between 0 and 0.99, preferably between 0 and 0.60.

The term “reflux ratio” means the mass ratio [organic phase (E)/stream (A)].

Thus, the process according to the invention makes it possible to obtain a dried methyl mercaptan as defined above.

The process according to the invention may be performed continuously or batchwise, preferably continuously.

During steps 1) to 7) of the process, the pressure may be between 0.05 and 75 bar absolute, preferably between 1 and 30 bar absolute, more preferentially between 5 and 15 bar absolute, for example approximately 10, 11, 12, 13, 14 or 15 bar absolute. Methanol, preferably in trace amounts, may be included in stream (A) and/or the distillate (B) and/or the condensate (C) and/or the aqueous phase (D) and/or stream (F).

According to one embodiment, stream (A) is connected to a unit for producing methyl mercaptan from methanol and hydrogen sulfide.

According to one embodiment, stream (A) is connected to a unit for producing methyl mercaptan from at least one carbon oxide, hydrogen and hydrogen sulfide and/or sulfur.

The present invention also relates to the use of azeotropic distillation for drying methyl mercaptan. In particular, said azeotropic distillation corresponds to the distillation as described for step 2) of the drying process according to the invention.

The present invention also relates to the dried methyl mercaptan as defined above.

Process for Preparing Methyl Mercaptan Via the Methanol Route

The present invention also relates to a process for producing methyl mercaptan, comprising the following steps:
a) reacting methanol with hydrogen sulfide to form a stream (M), preferably in gaseous form, comprising methyl mercaptan, water, possibly unreacted H₂S and sulfur byproducts;
b) optionally, said stream (M) is condensed;
c) optionally, at least one step of purification of said stream (M) is performed to obtain a stream enriched in methyl mercaptan; and
d) the stream obtained in step a), b) or c) is dried via the drying process as described above.

Preferably, in step c), said at least one purification step corresponds to at least one step of phase separation, preferably by decantation, and/or to at least one distillation step.

Step c) may notably correspond to one or more phase separation steps, for example one or two decantation steps, and/or one or more distillation steps, for example one or two distillation steps.

In particular, after step c), a stream enriched in methyl mercaptan and comprising water is obtained.

Preferably, step c) makes it possible, via one or more purification steps, to remove from stream (M) the H₂S, the sulfur byproducts and the majority of the water.

Preferably, at least 50% by weight of the water, for example at least 70%, or even at least 90% by weight of the water is removed from stream (M) by virtue of step c). The H₂S and the sulfur byproducts may remain in trace amounts on conclusion of step c). Said process may thus comprise the following steps:
a) methanol is reacted with hydrogen sulfide to form a stream (M) comprising methyl mercaptan, water, unreacted H₂S and sulfur byproducts;
b) optionally, said stream (M) is condensed;
c1) the following are separated out, preferably by decantation, from stream (M):
   a gas stream (N) comprising unreacted hydrogen sulfide;
   an aqueous stream (O); and
   a stream (P) comprising methyl mercaptan, water, unreacted hydrogen sulfide and sulfur byproducts;
c2) stream (P) is distilled so as to obtain:
   a stream (R) comprising hydrogen sulfide, preferably at the top of the column; and
   a stream (S) comprising methyl mercaptan, water and sulfur byproducts, preferably at the bottom of the column;
c3) stream (S) is distilled so as to obtain:
   a stream (T) comprising methyl mercaptan and water, preferably at the top of the column; and
   a stream (U) comprising the sulfur byproducts, preferably at the bottom of the column;
c4) optionally, the methyl mercaptan of stream (T) is separated from the water, preferably by decantation, so as to obtain:
   a stream (V) comprising methyl mercaptan and water; and
   a stream (W) comprising water;
d) stream (T) or (V) is dried via the drying process according to the invention.

Thus, steps c1 to c4 are purification steps for obtaining streams that are increasingly rich in methyl mercaptan.

Said streams (M) and/or (P) and/or (S) and/or (T) and/or (V) may possibly comprise unreacted methanol, preferably in trace amounts.

Step a)—Reaction:

In step a), methanol is reacted with hydrogen sulfide to form a stream (M) comprising methyl mercaptan, water, possibly unreacted H₂S and possibly sulfur byproducts.

Prior to step a), a gaseous stream of the H₂S and methanol reagents may be prepared as follows.

Liquid methanol is injected into gaseous H₂S. This injection enables the methanol to be partially or totally vaporized. The mixture of H₂S and methanol can then be totally vaporized, if necessary, so as to obtain a totally gaseous stream.

Thus, a gas stream of H₂S and methanol, preferably prepared as above, or separately methanol and H₂S, each in gaseous form, are introduced into a reactor.

Said reactor may be isothermal or adiabatic, with plates, multi-tubular or with a fixed bed. An adiabatic reactor is preferably chosen.

The reaction temperature may be between 200° C. and 500° C., preferably between 200° C. and 400° C. Preferably, the reaction temperature is between 200° C. and 360° C.

Above this temperature, the catalyst may be physically damaged (notably by sintering and coking).

The pressure may be between 1 and 40 bar absolute.

The H₂S/methanol mole ratio may be between 1 and 50, preferably between 1 and

The H₂S is preferably in excess relative to methanol.

The reactor may contain a catalyst for the methyl mercaptan formation reaction, preferably in the gas phase. Among the catalysts that may be used, mention may be made of:
alumina-based catalysts;
thorium dioxide ThO₂, preferably deposited on a silicate support;
catalysts based on cadmium sulfide, preferably on an alumina support;
catalysts based on the following oxides: MgO, ZrO₂, rutile (R) and anatase (A) TiO₂, CeO₂, and γ-Al₂O₃;
catalysts based on metal oxides, preferably doped with alkali metals (Li, Na, K, Rb, Cs) and optionally supported on SiO₂, Al₂O₃ or Nb₂O₅;
catalysts based on alkali metal carbonates;
catalysts based on alkali metal salts with certain acids of transition metals (Cr, Mo, W, Ni), impregnated on γ-alumina or other metal oxides;
potassium tungstate on alumina K₂WO₄/Al₂O₃.

A stream (M) is thus obtained comprising alkyl mercaptan, water, possibly unreacted H₂S and sulfur byproducts.

Step b)—Condensation:

Stream (M) obtained on conclusion of step a) may optionally be condensed by means of any conventional technique, preferably using one or more condensers or economizers. During condensation, stream (M) is notably cooled as low as possible, to maximize the removal of water, but must be maintained strictly above 16° C. to avoid the formation of solid hydrates of methyl mercaptan. Preferably, stream (M) is condensed at a temperature of between 20° C. and 70° C., for example between 30° C. and 60° C.

Step c)—Purification:

Preferably, in step c), said at least one purification step corresponds to at least one step of phase separation, preferably by decantation, and/or to at least one distillation step. Step c) may notably correspond to one or more phase separation steps, for example one or two decantation steps, and/or one or more distillation steps, for example one or two distillation steps.

Preferably, step c) makes it possible, via one or more purification steps, to remove from stream (M) the unreacted H₂S and/or the sulfur byproducts and/or the water. In particular, after step c), a stream enriched in methyl mercaptan is obtained.

The purification step c) may be performed via any conventional technique and in particular according to steps c1) to c4) as described below.

Step c1—Separation:

In the separation step c1), preferably by decantation, the following are obtained:
a gas stream (N) comprising unreacted hydrogen sulfide;
an aqueous stream (O); and
a stream (P) comprising methyl mercaptan, water, unreacted hydrogen sulfide and sulfur byproducts.

Preferably, stream (M) is separated at a temperature of between 20° C. and 70° C., preferably between 30° C. and 60° C. The pressure may be between 1 and 40 bar absolute.

Stream (P) obtained may notably be in gaseous form or in liquid form. When stream (P) is in gaseous form, streams (N) and (P) may be combined.

In particular, the aqueous stream (O), preferably in liquid form, comprises at least 50%, preferably at least 70%, more preferentially at least 90% by weight of water, relative to the total weight of the water present in stream (M). The aqueous stream (O) may thus be sent to a degasser. The degassed aqueous stream may then be sent for waste water treatment.

The gas stream (N) may be recycled into the reactor feed for step a). In this case, purging of this stream (N) is performed so as to avoid the accumulation of inert matter and/or impurities in this recycling loop. Examples of inert matter and/or impurities that may be mentioned include: methane, CO, CO₂, H₂ and N₂. The gas stream resulting from this purging is called vent E1. When streams (N) and (P) are combined, the same type of purging may be performed so as to obtain a gas stream called vent E1′.

According to one embodiment, the vents E1 or E1′ are sent for incineration.

According to another embodiment, the vents E1 or E1′ can be sent to a methanol absorption column, in order to recover the sulfur compounds such as H₂S and/or methyl mercaptan which they comprise, by gas(vent)-liquid(methanol) extraction.

Step c2— Removal of H₂S by Distillation:

Distillation of stream (P) is then be performed so as to obtain:
a stream (R) comprising hydrogen sulfide, preferably at the top of the column; and
a stream (S) comprising methyl mercaptan, water and sulfur byproducts, preferably at the bottom of the column;

During the distillation, the pressure may be between 1 and 40 bar absolute, and/or the temperature may be between −60° C. and +60° C., at the top of the column, and between +20° C. and +200° C. at the bottom of the column.

Stream (R) comprising H₂S may be recovered at the top of the column, and optionally recycled into the reactor feed for step a).

In particular, said distillation of step c2) makes it possible to remove the H₂S remaining in stream (P) (it is understood that traces of H₂S may remain in stream (S)).

Step c3—Removal of the Sulfur Byproducts by Distillation:

Distillation of stream (S) is performed so as to obtain:
a stream (T) comprising methyl mercaptan and water, preferably at the top of the column; and
a stream (U) comprising the sulfur byproducts, preferably at the bottom of the column.

During the distillation, the pressure may be between 1 and 40 bar absolute, and/or the temperature may be between +20° C. and +100° C., at the top of the column, and between +40° C. and +200° C. at the bottom of the column.

In particular, said distillation of step c3) makes it possible to remove the sulfur byproducts remaining in stream (S) (it is understood that traces of the sulfur byproducts may remain in stream (T)).

Step c4— Separation of Methyl Mercaptan and Water:

Prior to step c4), stream (T) can be cooled as low as possible, to maximize the water removal, but must be kept strictly above 16° C. to avoid the formation of solid hydrates of methyl mercaptan. Preferably, stream (T) is cooled to a temperature of between and 70° C., for example between 30° C. and 60° C.

This cooling makes it possible to maximize the separation of water during step c4), while maintaining a temperature strictly above 16° C. to avoid the formation of solid hydrates of methyl mercaptan.

Separation of the methyl mercaptan and of the remaining water can then be performed, preferably by decantation, so as to obtain:
a stream (V) comprising methyl mercaptan and water, preferably in liquid form;
a stream (W) comprising water, preferably in liquid form.

In particular, in step c4), stream (W) comprises at least 50% by weight, preferably at least 70%, more preferentially at least 90% by weight of water, relative to the total weight of the water present in stream (T).

Stream (T) or stream (V) notably corresponds to stream (A) as defined above.

Stream (V) or stream (T) obtained can then be dried according to the drying process according to the invention.

In the separation step c4), it is possible to recover the gas phase thus separated from streams (W) and (V), which are both in liquid form. This gas stream is called vent E2.

According to one embodiment, the vents E2 are incinerated.

According to another embodiment, the vents E2 can be sent to a methanol absorption column, in order to recover the sulfur compounds such as H₂S and/or methyl mercaptan which they comprise, by gas(vent)-liquid(methanol) extraction.

Process for Preparing Methyl Mercaptan Via the Carbon Oxide(s) Route

The process for producing methyl mercaptan via the carbon oxide(s) route is performed using at least one carbon oxide, hydrogen and hydrogen sulfide and/or sulfur. The carbon oxide is chosen from carbon monoxide (CO) and carbon dioxide (CO₂). Preferably, the carbon oxide is carbon monoxide (CO).

Said process is thus preferentially performed using a mixture of carbon monoxide, hydrogen and hydrogen sulfide. The main byproduct of this synthesis is carbon dioxide (CO₂).

Carbon oxysulfide (COS) is considered as the reaction intermediate which leads to methyl mercaptan after hydrogenation according to the following reactions:

CO+H₂S→COS+H₂

COS+3H₂→CH₃SH+H₂O

CO₂ is itself the result of several side reactions such as:

CO+H₂O→CO₂+H₂

COS+H₂O→CO₂+H₂S

2 COS→CO₂+CS₂

The carbon dioxide obtained can optionally be recycled to also produce methyl mercaptan, according to the following equation:

CO₂₊₃H₂+H₂S→CH₃SH+2H₂O

Such a process for producing methyl mercaptan is widely described, for instance in patent applications EP 0171312 or WO 08/125452.

The present invention thus relates to a process for producing methyl mercaptan, comprising the following steps:
a-ox) at least one carbon oxide, H₂, H₂S and/or sulfur are reacted, preferably in gaseous form, in the presence of at least one catalyst in order to form a stream (J), preferably in gaseous form, comprising methyl mercaptan, water and possibly said at least one carbon oxide, H₂, unreacted H₂S and carbonyl sulfide (COS);
b-ox) said stream (J) is optionally condensed;
c-ox) optionally, at least one purification step of said stream (J) is performed to obtain a stream enriched in methyl mercaptan; and
d-ox) the stream obtained in step a-ox), b-ox) or c-ox) is dried via the drying process as defined above.

When the carbon oxide is CO, stream (J) may comprise unreacted CO and CO₂ formed during step a-ox).

In particular, said process for producing methyl mercaptan comprises the following steps:
a-ox) at least one carbon oxide, H₂, H₂S and/or sulfur are reacted, preferably in gaseous form, in the presence of at least one catalyst in order to form a stream (J), preferably in gaseous form, comprising methyl mercaptan, water and said at least one carbon oxide, H₂, unreacted H₂S and carbonyl sulfide (COS);
b-ox) said stream (J) is condensed;
c1-ox) the following are separated out, preferably by decantation, from the liquid stream (J):
   a liquid organic phase (K) comprising methyl mercaptan and water; and
   a liquid aqueous phase (L);
c2-ox) optionally, separation of the incondensable compounds is performed, so as to obtain a stream (J′), preferably in gaseous form; said separation being able to be performed simultaneously with step b-ox or with step c1-ox);
d-ox) stream (K) is dried according to the drying process as defined above; and e-ox) stream (J′) is optionally recycled into step a-ox).

Thus, steps c1-ox and c2-ox are notably purification steps to obtain streams that are increasingly rich in methyl mercaptan.

According to one embodiment, stream (J) or stream (K) corresponds to stream (A) according to the invention.

Step a-ox)—Reaction:

Reaction step a) is well known. In particular, step a-ox) is performed at a temperature of between 200° C. and 500° C., preferably between 200° C. and 400° C. In particular, step a-ox) is performed at a pressure of between 1 and 100 bar absolute, preferably between 3 and 30 bar absolute.

Preferably, in step a-ox), the carbon oxide/S/H₂S/H₂ mole ratio is between 1/0/0.05/0.05 and 1/20/40/100. It is preferably between 1/0/0.5/1 and 1/0/10/20. In particular, it is 1/0/1/2.

Preferably, in step a-ox), in the absence of sulfur, the CO/H₂/H₂S ratio is between 1/0.05/0.05 and 1/40/100. It is preferably between 1/0.5/1 and 1/10/20. In particular, it is 1/2/1.

Step a-ox) may be performed on one or more catalytic beds, which are preferably fixed beds. It may be performed in a reactor comprising one or more reaction zones, the reagent(s) possibly being fed in between the various zones. Thus, the reagents, preferably H₂ and/or H₂S, may be introduced separately onto the various catalytic beds or reaction zones.

Said at least one catalyst used in step a-ox) is known and may notably be chosen from:
catalysts based on molybdenum and potassium supported on zirconia such as K₂MoO₄/ZrO₂, as described in WO 2019/122072.
These catalysts are tested at a temperature of 320° C. and at a pressure of 10 bar using a CO/H₂/H₂S ratio of 1/2/1.
catalysts based on molybdenum and potassium of Mo—S—K and/or Mo—O—K type on a hydroxyapatite support such as K₂MoS₄/Ca₁₀(PO₄)₆(OH)₂ or K₂MoO₄/Ca₁₀(PO₄)₆(OH)₂ as described in WO 2014/154885. These catalysts are tested at a temperature of 280° C. and at a pressure of 10 bar using a CO/H₂/H₂S ratio of 1/2/1.
the catalysts described in patent application US 2010/0286448, composed of a porous support such as SiO₂, TiO₂, silica-aluminas, zeolites and carbon nanotubes, onto which a metal has been electrolytically deposited. K₂MoO₄, and also another metal oxide acting as promoter, are then impregnated onto this support.
catalysts based on Mo and K (notably K₂MoO₄) promoted with TeO₂ and supported, such as K₂MoO₄/TeO₂/SiO₂, described in US 2010/094059.
The catalyst K₂MoO₄/TeO₂/SiO₂ is tested for a temperature of 300° C. and at a pressure of 2 bar, taking a CO/H₂/H₂S ratio of 1/1/2 and an hourly space velocity of 2000 h⁻¹.
International patent application WO 2005/040082 describes several catalysts and notably a catalyst comprising an active component based on Mo—O—K, an active promoter and optionally a support. The catalysts illustrated are K₂MoO₄/Fe₂O₃/NiO or K₂MoO₄/CoO/CeO₂/SiO₂, each supported on silica. These catalysts are tested at a temperature of 320° C. and at a pressure of 7 bar, taking a CO/H₂/H₂S ratio of 1/1/2 and an hourly space velocity of 3000 h⁻¹.

Step b-ox)—Condensation:

Any type of condenser may be used for this operation, such as tubular or plate exchangers. Preferably, the condenser has separated fluids, i.e. there is no contact between the gases to be condensed and the refrigerant fluid. The refrigerant fluid may be liquid or gaseous such as air, water, brine, ammonia, freons, oils, etc.

The condensation temperature may be between 20° C. and 70° C., preferably between 30° C. and 60° C. The pressure may be between 1 bar absolute and 100 bar absolute. The object is to condense a maximum amount of methyl mercaptan and water relative to the uncondensable compounds (such as CO/COS/CO₂/H₂/H₂S), which will allow easy separation of the liquid and gas phases.

Step c-ox)—Purification:

Step c1-Ox)—Water Separation

The separation step c1-ox) may be performed via any conventional technique and in particular by decantation. Preferably, stream (J) is in liquid form. Thus, the following are separated out, preferably by decantation, from stream (J):
an organic phase (K) comprising methyl mercaptan and water; and
an aqueous phase (L).

In particular, in step c1-ox), the aqueous phase (L) comprises at least 50% by weight, preferably at least 70%, more preferentially at least 90% by weight of water, relative to the total weight of the water present in stream (J).

Step c2-Ox—Separation of the Incondensable Compounds:

The term “uncondensable compounds” notably means the compounds which remain in gaseous form at the temperatures and pressures of said production process, notably after the condensation step b-ox). Mention may notably be made, as incondensable compounds, of carbon oxide (CO and/or CO₂), H₂, H₂S, carbonyl sulfide (COS), methane and any other inert incondensable compound produced or introduced during said process.

The separation may be performed via any conventional technique. A stream (J′) is notably obtained in gaseous form comprising incondensable compounds such as carbon oxide (CO and/or CO₂), H₂, H₂S, carbonyl sulfide (COS), methane and any other inert incondensable compound produced or introduced during said process.

According to one embodiment, stream (J′) can be recycled into step a-ox), preferably directly (without an intermediate purification step). According to another embodiment, stream (J′) can be partly purged. If it is not recycled, it can be sent to the incinerator or any other gas treatment unit.

In particular, whether the process for producing methyl mercaptan is via the methanol route or via the carbon oxide(s) route, these routes may each comprise at least one purification step as defined above which is a step for separating the water and methyl mercaptan prior to drying, and preferably by decantation (for example step c1 and/or c4 and c1-ox, respectively).

In particular, such a step makes it possible to separate the water from the methyl mercaptan so as to obtain a methyl mercaptan with a residual water content, that is to say to obtain a methyl mercaptan with a water content which depends on the solubility of water in methyl mercaptan, at the separation temperature. Generally, this content is between 0.15%, preferably strictly greater than 0.15%, and 30% by weight of water relative to the total by weight of methyl mercaptan and water, for example between 0.15% and 10%. Preferably, it is between 0.15%, preferably strictly greater than 0.15%, and 5% by weight of water relative to the total by weight of methyl mercaptan and water. For example, it is between 0.15%, preferably strictly greater than 0.15%, and 2%, for example between 0.15% and 1.5% or between 0.15% and 1% of water, relative to the total by weight of methyl mercaptan and water.

Following this step, the process for drying methyl mercaptan as described above may be implemented more efficiently and economically, the amount of water before drying having been reduced to a minimum.

### DESCRIPTION OF THE FIGURES

FIG. 1:

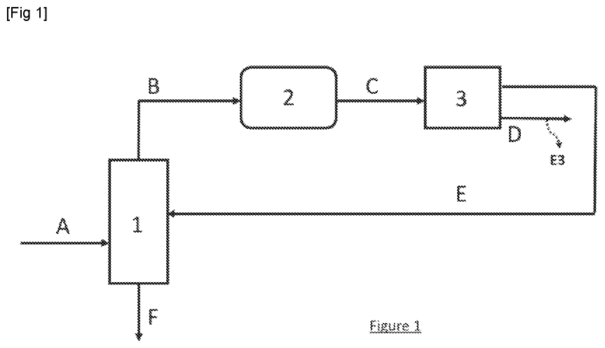

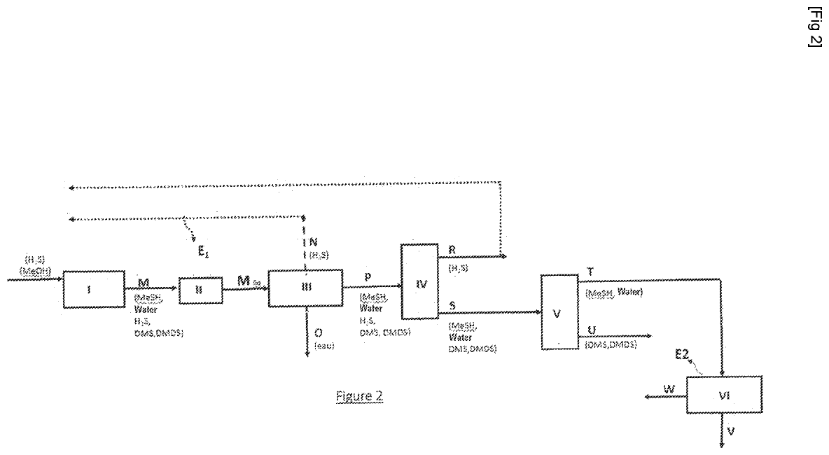

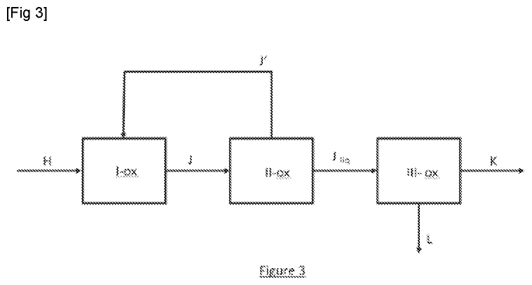

FIG. 1 shows one embodiment of the drying process according to the invention. Stream (A) enters distillation column (1). Stream (A) is distilled in column (1). The distillate (B) is recovered at the top of the column in gaseous form. The distillate (B) is then condensed in a condenser (2) where it is recovered in two-phase liquid form (condensate (C)). The condensate (C) then settles in the decanter (3) so as to obtain:
an aqueous phase (D); and
an organic phase (E).

The organic phase (E) then serves as reflux for the distillation column (1).

The dried methyl mercaptan is recovered at the bottom of column (1) (stream (F)).

FIG. 2:

FIG. 2 shows an embodiment of a process for producing methyl mercaptan via the methanol route.

The reaction step a) is performed in a reactor (I) using methanol and H₂S.

Stream (M) leaving the reactor (I) comprises MeSH, water, H₂S and sulfur byproducts.

Stream (M) is condensed in a condenser (II). It is then separated in a decanter (III) into three streams:
a stream (N) comprising H₂S;
a stream (O) comprising water; and
a stream (P) comprising MeSH, water, H₂S and sulfur byproducts.

Stream (P) is distilled in a distillation column (IV) to remove the H₂S (stream (R) at the top of the column) and to obtain a stream (S) at the bottom of the column comprising MeSH, water and sulfur byproducts. Stream (S) is then distilled in a distillation column (V) to obtain a stream (U) at the bottom of the column comprising the sulfur byproducts and a stream (T) at the top of the column comprising the MeSH and the water. Stream (T) is then separated in a decanter (VI) into a stream (V) comprising MeSH and water and a stream (W) comprising water.

FIG. 3:

FIG. 3 represents one embodiment of a process for producing methyl mercaptan via the carbon oxide route.

A stream (H) comprising CO, hydrogen and H₂S is introduced in gaseous form into a reactor I-ox so as to recover at the outlet a stream (J) comprising methyl mercaptan, water and possibly CO, CO₂, H₂, unreacted H₂S and carbonyl sulfide (COS).

Stream (J) is condensed and then separated in the condenser II-ox so as to obtain a stream (J) in liquid form enriched in methyl mercaptan and a stream (J′) in gaseous form comprising CO, CO₂, H₂, unreacted H₂S and carbonyl sulfide (COS);

Stream (J′) is then recycled into the reactor I-ox. The following are then separated in a decanter III-ox, from stream (J):
an organic phase (K) comprising methyl mercaptan and water; and
an aqueous phase (L).

Stream (K) is then dried according to the drying process of the invention.

The examples that follow illustrate the present invention but are not in any way limiting.

### EXAMPLES

### Example 1: Comparative Test with Molecular Sieves

Continuous drying on molecular sieves requires at least two dryers in parallel (the second in regeneration when the first is in adsorption).

An adsorption column (dryer) is used which contains 1 kg of Siliporite RA molecular sieves (⅛ inch in particle size); the flow rate of methyl mercaptan to be dried is 1 kg/h. The inlet and outlet compositions of this dryer are as follows:

TABLE 1

| | Inlet | Outlet |
|---|---|---|
| Dimethyl sulfide (DMS-ppm) | 153 | 550 |
| Water (ppm) | 3871 | 80 |
| Methyl mercaptan (MeSH-%) | 99.38 | 99.89 |
| Methanol (MeOH-ppm) | 1962 | 176 |
| TOTAL (%) | 99.98 | 99.97 |

When molecular sieves are used, it can be observed that the amount of dimethyl sulfide (DMS) has been multiplied by 3 after drying and the amount of methanol has been reduced almost tenfold. Indeed, the methanol is adsorbed onto the molecular sieves with the consequence of considerably reducing the water drying capacity of these sieves, and thus of increasing the frequency of the adsorption/regeneration cycles.

### Example 2: Drying Process According to the Invention

The drying process corresponds to that described for FIG. 1.

A stream of MeSH to be dried comprising 99.77% by weight of MeSH (1000 kg/h) and 0.23% by weight of water (2.3 kg/h), relative to the total weight of MeSH and water, is introduced into a distillation column.

The azeotropic distillation column comprises 28 trays and meets the following criteria:
the distillation pressure is 13 bar absolute;
the temperature profile is between 90° C. at the bottom and 70° C. at the top of the column;
the reflux ratio is 47%.

The distillate is recovered at the top of the column in gaseous form. It comprises 98.88% by weight of MeSH (467.4 kg/h) and 1.12% by weight of water (5.3 kg/h), relative to the total weight of MeSH and water (472.7 kg/h). The temperature of the distillate is about 72° C. for a pressure of about 12 bar absolute.

The distillate is then condensed in a condenser. Its composition remains identical and it is recovered in two-phase liquid form at a temperature of about 40° C. for a pressure of about 12 bar absolute. The condensate then settles in a decanter so as to obtain:
an aqueous phase comprising 98.26% (2.26 kg/h) by weight of water and 1.74% by weight of MeSH (0.04 kg/h) relative to the total weight of the aqueous phase (2.3 kg/h), and
an organic phase comprising 99.36% by weight of MeSH (467.5 kg/h) and 0.64% by weight of water (3 kg/h), relative to the total weight of MeSH and water (470.5 kg/h). The temperature is about 40° C. for a pressure of about 12 bar absolute for the two phases.

The dried MeSH is recovered at the bottom of the distillation column and contains less than 10 ppm by weight of water relative to the total by weight of methyl mercaptan and water.

The amounts of methanol and dimethyl sulfide are the same at the inlet of stream (A) as in the dried methyl mercaptan (about 0.04 kg/h and 0.1 kg/h, respectively).

Consequently, the process according to the invention makes it possible to effectively dry the methyl mercaptan while at the same time not increasing the amount of the DMS byproduct. Furthermore, the drying process is not affected by traces of methanol and may be performed continuously without its performance being impaired.

1. A process for drying methyl mercaptan, comprising the following steps:
1) a stream (A) comprising methyl mercaptan and water is introduced into a distillation column (1);
2) said stream (A) is distilled in said column (1);
3) the distillate (B) is recovered in gaseous form, preferably at the top of the column;
4) the distillate (B) is condensed, preferably in a condenser (2), so as to obtain a condensate (C) in liquid form;
5) said condensate (C) is separated, preferably using a decanter (3), so as to obtain two separate liquid phases:
an aqueous phase (D); and
an organic phase (E) comprising methyl mercaptan;
6) all or part of the organic phase (E) is optionally introduced into the distillation column (1) as reflux; and
7) a stream (F) comprising the dried methyl mercaptan is recovered, preferably at the bottom of the column (1).

2. The drying process according to claim 1, in which the distillation of step 2) is performed at a pressure of between 0.05 and 75 bar absolute, preferably between 1 and 30 bar absolute, more preferentially between 5 and 15 bar absolute.

3. The drying process according to claim 1, in which the distillation of step 2) is performed at a temperature of between 20° C. and 200° C., preferably between 60° C. and 100° C., more preferentially between 65° C. and 95° C.

4. The drying process according to claim 1, in which the distillation of step 2) is an azeotropic distillation.

5. The drying process according to claim 1, in which stream (A) comprises at least 90%, preferably at least 95%, more preferentially at least 98%, for example at least 98.5%, or even at least 99% by weight of methyl mercaptan, relative to the total by weight of methyl mercaptan and water.

6. The drying process according to claim 1, in which the amount of water in stream (F) is between 0 and 1500 ppm, preferably between 0 and 1000 ppm, more preferentially between 40 and 800 ppm, relative to the total by weight of methyl mercaptan and water.

7. The drying process according to claim 1, in which, when step 6) is performed, the reflux ratio is between 0 and 0.99, preferably between 0 and 0.60.

8. The drying process according to claim 1, in which stream (A) is connected to a unit for producing methyl mercaptan from methanol and hydrogen sulfide.

9. The drying process according to claim 1, in which stream (A) is connected to a unit for producing methyl mercaptan from carbon oxide, hydrogen (H₂), hydrogen sulfide (H₂S) and/or sulfur (S).

10. A process for producing methyl mercaptan, comprising the following steps:
a) reacting methanol with hydrogen sulfide to form a stream (M), preferably in gaseous form, comprising methyl mercaptan, water, and possibly unreacted H₂S and sulfur byproducts;
b) optionally, said stream (M) is condensed;
c) at least one step of purification of said stream (M) is performed to obtain a stream enriched in methyl mercaptan;
d) the stream obtained in step c) is dried via the drying process of claim 1.

11. The use of azeotropic distillation for drying methyl mercaptan.
